(19) United States
(12) Patent Application Publication
(10) Pub. No.: US 2009/0130052 A1
(43) Pub. Date: May 21, 2009

(54) Biocide, in particular an agent with fungicidal activity

(76) Inventor: Oskar Schmidt, Wien (AT)

Correspondence Address: KNOBBE MARTENS OLSON & BEAR LLP, 2040 MAIN STREET, FOURTEENTH FLOOR, IRVINE, CA, 92614, US

(21) Appl. No.: 11/791,730

(22) PCT Filed: Nov. 4, 2005
(86) PCT No.: PCT/AT2005/000433

§ 371 Date: Nov. 12, 2008

(30) Foreign Application Priority Data
Nov. 5, 2004 (AT) A1847/2004

(51) Int. Cl. A61K 31/765; C08G 67/00

(52) U.S. Cl. 424/78.37; 528/392

(57) ABSTRACT

The invention relates to a polymeric condensation product which can be obtained by reacting guanidine or its salt with an alkylene diamine and an oxyalkylene diamine. Said condensation product has biocidal, in particular fungicidal, activity and is usable as a plant protection product.

The invention relates to a polymeric condensation product with antimicrobial or microbicidal, respectively, in particular fungicidal activity which is usable as a plant protection product.

Especially in recent years, the development of new antimicrobial agents has been a race against the increasingly developing resistance of microorganisms. On the other hand, advanced active agents having a broader spectrum of activities with less undesirable side effects are constantly being searched for.

In EP-A-0 439 698 and in EP-A-0 439 699, solutions of polymeric guanidine salts having an increased biocidal activity are described. Said polymeric guanidine salts are obtained by reacting a diamine with chlorine cyanide and subsequent polymerization.

From WO 99/54291, polyhexamethylene guanidines are known which can be used as disinfectants due to their microbiocidal effect. These substances are produced by polycondensation of guanidine with an alkylene diamine, in particular hexamethylene diamine. Polycondensation is effected by mixing hexamethylene diamine and guanidine hydrochloride at a molar ratio of 1:1 and heating the same to 120° C. for 5 hours. Subsequently, the temperature is raised to 150° C. and the mixture is heated at said temperature for another 10 hours. The condensation product obtained exhibits good biocidal activity, whereas, however, the fungicidal activity is only weak.

From WO 01/85676, on the other hand, biocidal polymers are known which are produced by condensation of guanidine and an oxyalkylene diamine. These substances are less toxic than the substances known from WO 99/54291 but fail to exhibit any fungicidal activity. For preparing said compound, for example, guanidinium hydrochloride is dissolved in triethylene glycol diamine at 50° C. Subsequently, this is heated to 120° C. and stirred for two hours at said temperature. Thereafter, said temperature is maintained for 2 hours, then a vacuum (0.1 bar) is applied and stirring is continued under vacuum for two more hours at 170° C. Subsequently, this is aerated to normal pressure, allowed to cool to 120° C. and diluted with demineralized water to approx. 50%. It is neutralized to a pH1 of 6 with phosphoric acid, allowed to cool and diluted to the desired concentration. The molecular weight was determined to be 1000 D.

The drug substance poly-[2-(2-ethoxyethoxyethyl)guanidinium hydrochloride] exhibits favourable pharmacodynamic properties, along with low toxicity and good tolerance from a pharmacological point of view, and can also be used as a medicine in antimicrobial therapy. The drug substance exhibits in particular an excellent antimicrobial activity which could be demonstrated by tests performed on a plurality of microorganisms such as multiresistant bacteria (which are resistant against common antibiotics), fungi (blastomyces, dermatophytes, mould fungi) and viruses such as Herpes simplex. Due to the quick microbicidal activity, a development of resistance can hardly be expected, as shown also by tests performed on a comparatively large number of bacterial strains.

It is the object of the invention to provide a new biocidal agent which, in particular, has a pronounced fungicidal activity, whereby, at the same time, toxicity should be low.

Said object is achieved by a polymeric condensation product which is obtainable by reacting guanidine or a salt thereof with an alkylene diamine and an oxyalkylene diamine.

It has turned out that the “mixed polycondensate” obtained from alkylene diamine and oxyalkylene diamine solves the problem posed according to the invention: it possesses biocidal, in particular fungicidal, activity, while, at the same time, exhibiting low toxicity.

In the reaction, preferably 0.8 to 1.2 moles of guanidine or a salt thereof, respectively, are used per mole of diamine (sum of alkylene diamine and oxyalkylene diamine).

The alkylene diamine and the oxyalkylene diamine are used especially at a molar ratio of between 4:1 and 1:4.

Moreover, diamines (alkylene diamine or oxyalkylene diamine, respectively) with terminal amino groups are preferably used.

A further preferred embodiment of the condensation product according to the invention is characterized in that a compound of general formula

NH₂(CH₂)ₙNH₂

is provided as the alkylene diamine, wherein n is an integer between 2 and 10, in particular 6, and that a compound of general formula

NH₂[(CH₂)₂O)]ₙ(CH₂)₂NH₂

is provided as the oxyalkylene diamine, wherein n is an integer between 2 and 5, in particular 2.

A hydrochloride has proven to be a suitable guanidine salt.

Furthermore, triethylene glycol diamine (relative molecular mass: 148), polyoxypropylene diamine (relative molecular mass: 230) and polyoxyethylene diamine (relative molecular mass: 600) are perfectly suitable as the oxyalkylene diamine.

The average molecular mass of the condensation product according to the invention preferably ranges between 500 and 3,000.

Furthermore, the invention relates to a biocide, in particular an agent with fungicidal activity, which comprises the polymeric condensation product according to the invention.

It has turned out that the polymeric condensation product according to the invention can be used very well as a plant protection product for the treatment of agricultural crops.

Using the agent according to the invention, for example, the fungus disease Black Sigatoka (pathogen: Mycosphaerella fijiensis var. differmis (MFD)) occurring in bananas can be controlled.

As a plant protection product, the agent can be used alone or together with inorganic or organic auxiliary agents.

The preparation of preferred representatives of the compounds used according to the invention is described below.

### EXAMPLE 1

Obtaining a (water-soluble) polycondensate from 1 mole of guanidine hydrochloride and a mixture of 0.5 mole of hexamethylene diamine and 0.5 mole of triethylene glycol diamine

74 g (0.5 mole) of liquid triethylene glycol diamine (relative molecular mass: 148) and 58 g (0.5 mole) hexamethylene diamine (relative molecular mass: 116) are introduced at room temperature into a three-necked flask equipped with a mechanical mixer and an air cooler and having a capacity of 100 ml. Subsequently, 96.5 g (1 mole) of powdered guanidine hydrochloride (relative molecular mass: 96.5), i.e., with a molar ratio of the reagents of 1:0.5:0.5, are added into the flask. The flask with the reaction mass was dipped into an oil bath with a temperature controller. The reaction mass was heated to 140° C. under constant stirring within 1 hour and subsequently was kept under stirring for one hour. Thereafter, the temperature was raised to a temperature of 170° C. under stirring within one hour and was kept at said temperature for another hour.

Subsequently, a vacuum was carefully applied and the reaction mass was stirred for another hour at 170° C. under a reduced pressure. From the first minutes of thermostatting as indicated, an intense release of ammonia (colouration of the indicator paper) occurred, demonstrating the progress of the reaction of polycondensation. In the course of the reaction, the reaction mass becomes viscous, and foam formation can thereby be observed visually. The reaction mixture gradually transforms into a resin of a yellowish colour. Once the gas release of the ammonia has been finished almost completely, the reaction is completed.

After cooling the flask, the polymer resin is removed from the flask with the aid of a spatula and is crushed to a powder in a mortar, which powder quickly dissolves in water. Approx. 190 g of the polymeric final product were obtained as a result of the experiment, this corresponds to a theoretical yield of 98.2%. The characteristic viscosity of the obtained polymer was measured: 0.4 N in an aqueous sodium chloride solution at 25° C. The viscosity is [η]=0.052 dl/g.

Data of the elemental analysis of the obtained polymer:

### C: 42.81%

### N: 21.40%

### H: 8.31%

This analysis points to a polycondensate with the total molecular formula C₇N₃OClH₁₆.

A new polymeric product was thus obtained which, in terms of its composition, corresponds to a co-polycondensate of guanidine hydrochloride and triethylene glycol diamine as well as hexamethylene diamine (at a ratio of 1:1).

It has turned out that said polycondensate is of minor toxicity (oral dose for rats LD₅₀>2000 mg/kg), whereby it has a substantially lower toxicity than PHMG (polyhexamethylene guanidine hydrochloride). Furthermore, it has been shown that it has a very high biocidal, in particular fungicidal, activity, whereby the fungicidal effect was demonstrated, among other things, by tests performed on the fungi Candida albicans and Aspergillus niger.

### EXAMPLE 2

Obtaining a water-soluble polycondensate from 1 mole of guanidine hydrochloride and a mixture of 0.75 mole of hexamethylene diamine and 0.25 mole of triethylene glycol diamine

If, in Example 1, the amount of hexamethylene diamine used is increased to 87 g (0.75 mole) while decreasing the amount of triethylene glycol diamine used to 37 g (0.25 mole), a polymer, which likewise is readily soluble in water, is obtained in an amount of 181 g, corresponding to 97% in theory, with the mode of operation otherwise being identical.

The characteristic viscosity of the obtained polymer was measured: 0.4 N in an aqueous sodium chloride solution at 25° C. is [η]=0.052 dl/g.

Elemental analysis of the obtained polymer:

### C: 44.7%

### N: 22.91

### H: 8.52%.

A new polymeric product was obtained which, in terms of its composition, corresponds to a co-polycondensate of guanidine hydrochloride and triethylene glycol diamine as well as hexamethylene diamine (at a ratio of 1:3).

It has turned out that said polycondensate is of minor toxicity (oral dose for rats LD₅₀>2000 mg/kg), whereby it has a substantially lower toxicity than PHMG (polyhexamethylene guanidine hydrochloride). Furthermore, it has been shown that it has a very high biocidal, in particular fungicidal, activity, whereby the fungicidal effect was demonstrated, among other things, by tests performed on the fungi Candida albicans and Aspergillus niger.

1. A polymeric condensation product, obtainable by reacting guanidine or a salt thereof with an alkylene diamine and an oxyalkylene diamine.

2. A condensation product according to claim 1, obtainable by a reaction in which 0.8 to 1.2 moles of guanidine or a salt thereof, respectively, are used per mole of diamine (sum of alkylene diamine and oxyalkylene diamine).

3. A condensation product according to any of claims 1 or 2, obtainable by a reaction in which the alkylene diamine and the oxyalkylene diamine are used at a molar ratio of between 4:1 and 1:4.

4. A condensation product according to any of claims 1 to 3, characterized in that the amino groups of the alkylene diamine and/or the oxyalkylene diamine are terminal.

5. A condensation product according to any of claims 1 to 4, characterized in that a compound of general formula
NH₂(CH₂)ₙNH₂
is provided as the alkylene diamine, wherein n is an integer between 2 and 10, in particular 6.

6. A condensation product according to any of claims 1 to 5, characterized in that a compound of general formula
NH₂[(CH₂)₂O)]ₙ(CH₂)₂NH₂
is provided as the oxyalkylene diamine, wherein n is an integer between 2 and 5, in particular 2.

7. A condensation product according to any of claims 1 to 6, characterized in that a hydrochloride is provided as the salt of the guanidine.

8. A condensation product according to any of claims 1 to 5 and 7, characterized in that triethylene glycol diamine (relative molecular mass: 148), polyoxypropylene diamine (relative molecular mass: 230) and/or polyoxyethylene diamine (relative molecular mass: 600) are provided as the oxyalkylene diamine.

9. A condensation product according to any of claims 1 to 8, characterized in that its average molecular mass ranges between 500 and 3,000.

10. A biocide, in particular an agent with fungicidal activity, comprising a polymeric condensation product according to any of claims 1 to 9.

11. The use of a polymeric condensation product according to any of claims 1 to 9 for the treatment of agricultural crops.

12. The use according to claim 11, characterized in that the agricultural crops to be treated are bananas.

13. The use according to claim 12 for controlling the fungus disease Black Sigatoka.
